(19) United States
(12) Patent Application Publication
(10) Pub. No.: US 2024/0277288 A1
(43) Pub. Date: Aug. 22, 2024

(54) Wearable, voice and touch-controlled device for tracking, reminding/alerting, correlating, predicting, suggesting, documenting, integrating, and transmitting postnatal and postpartum care and development events

(71) Applicant: Sheena A. Young, Clarksburg, MD (US)

(72) Inventor: Sheena A. Young, Clarksburg, MD (US)

(21) Appl. No.: 18/584,037

(22) Filed: Feb. 22, 2024

Related U.S. Application Data
(60) Provisional application No. 63/447,576, filed on Feb. 22, 2023.

(51) Int. Cl. A61B 5/00; A61B 5/0205; G16H 20/30; G16H 40/20; G16H 40/67

(52) U.S. Cl. CPC A61B 5/486; A61B 5/0205; A61B 5/681; A61B 5/7246; A61B 5/7267; A61B 5/7275; A61B 5/7435; A61B 5/746; A61B 5/749; G16H 20/30; G16H 40/20; G16H 40/67; A61B 2503/04; A61B 2503/10

(57) ABSTRACT

A wearable device that uses voice recognition/control and touch, with machine learning and predictive analytics, to monitor the occurrence and duration of postnatal and postpartum care and development events. The device will track, remind/alert, correlate, predict, suggest, document, integrate and transmit past, present, and future events to alleviate the burden that users experience with management by manual or less comprehensive methods.

### FEDERALLY SPONSORED RESEARCH

Not Applicable

### SEQUENCE LISTING OR PROGRAM

Not Applicable

### TECHNICAL FIELD OF THE INVENTION

The present invention relates generally to wearable smartwatches and fitness or event trackers. More specifically, the present invention relates to a wearable, voice and touch-controlled device for tracking, reminding/alerting, correlating, predicting, suggesting, documenting, integrating and transmitting postnatal and postpartum care and development events.

### BACKGROUND OF THE INVENTION

For new parents and those on second, third, or more children, taking care of a newborn is exciting but bewildering. There are endless thoughts in the mind of parents; is the baby sleeping or eating properly, is the baby reaching developmental milestone on time? Sleep-deprived and stressed-out parents can not trust their memory to remember everything.

As such a number of companies offer tracking materials with their products, such as diaper manufacturers, baby formula and supplement companies, and various doctor offices and medical practices. These tracking materials can come in the form of papers, hanging charts, phone applications, or even basic electronic devices.

Parents strive hard for being connected to their newborn and ensuring their newborn gets the required care. While app development companies are coming up with baby trackers in today's current digital and big data environment. For example, “Glow Baby App” reports it has helped with 200,000 pregnancies and reached 3 million users after only being on the market since 2016.

Many studies, such as Effects of Social Media and Mobile Health Apps on Pregnancy Care: Meta-Analysis, suggest health-tracking apps can be helpful for pregnant people, but not everyone responds positively. In one survey, Understanding the Role of Healthy Eating and Fitness Mobile Apps in the Formation of Maladaptive Eating and Exercise Behaviors in Young People, almost half of the participants said they had negative experiences with these apps, including feelings of guilt and fear, which are feelings are already heightened in new parents.

Therefore, what is missing in the baby tracking market place is the combination of wearable electronic devices for both the mother and the mother and baby which can use artificial intelligence (AI) or algorithms to automatically and non-intrusively monitor and track both the mother's daily habits as well as the baby's daily habits, using a wearable device with sensors for obtaining biometric and other activity data, recording the data, saving the data, and using the data to create tracking results, identify problems, concerns or if/when goals are or are not being met for both the mother and the baby/newborn, and suggest adjustments/solutions.

It is an objective of the invention to teach a wearable, voice and touch-controlled device for tracking, reminding/alerting, correlating, predicting, suggesting, documenting, integrating and transmitting postnatal and postpartum care and development events that minimizes the required interaction and manual input of data or tracking information, as well as reduces the potential for general and expected user error in manual tracking or mental health disadvantages of having to obsessively interact with a device or chart to ensure information and data is being entered and tracked accurately. While the wearable device teaches sensors and automatic detection and collection of data as input, there are also alternative options for hands-free input.

### SUMMARY OF THE INVENTION

The present invention is a wearable device that uses voice recognition/control and touch, with machine learning and predictive analytics, to monitor the occurrence and duration of postnatal and postpartum care and development events. The device will track, remind/alert, correlate, predict, suggest, document, integrate and transmit past, present, and future events to alleviate the burden that users experience with management by manual or less comprehensive methods, although there is manual input through touch or voice.

The device can be controlled by voice, which is ideal when caring for a baby where the caregiver is often placed in situations with limited hand mobility such as diaper changing, feeding, or even playing.

Machine Learning/Predictive Analytics provides activity recognition and serves to correlate baby and mom/dad data to better predict events using statistical analysis/trending techniques. On a general level, the present invention can also be used for correlations of other multi-person relationships.

In a general workflow taught by the present invention, mother and baby behavioral/physiological data is inputted to the wearable device(s) via voice or touch input and/or collection via a sensor. The individual data is then processed and integrated together as well as individually being sent to a data correlation/trending module for analysis. The baby and mother can each have their own data correlation/trending module for analysis using machine learning (ML) which generates specific, individual observed relationship and suggested behavioral change information. The integrated data also undergoes analysis through a separate data correlation and trending module, using machine learning (ML). The correlated/trending integrated data as well as the observed relationship and suggested behavioral changes for both the mother and baby are finally combined into an observed mother/baby relationship and suggested behavioral changes output.

### BRIEF DESCRIPTION OF THE DRAWINGS

The accompanying drawings, which are incorporated herein a form a part of the specification, illustrate the present invention and, together with the description, further serve to explain the principles of the invention and to enable a person skilled in the pertinent art to make and use the invention.

FIG. 1 is a flow chart of an exemplary machine learning (ML) workflow taught by the present invention.

FIG. 2 is a flow chart of a general workflow taught by the present invention.

FIG. 3 is a flow chart of a generic workflow taught by the present invention.

FIG. 4 is a flow chart of a specific workflow taught by the present invention illustrating one exemplary mode of operation.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized, and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

Now referring to the Figures, the present invention is a wearable, voice and touch-controlled device for tracking, reminding/alerting, correlating, predicting, suggesting, documenting, integrating and transmitting postnatal and postpartum care and development events that minimizes the required interaction and manual input of data or tracking information, as well as reduces the potential for general and expected user error in manual tracking or mental health disadvantages of having to obsessively interact with a device or chart to ensure information and data is being entered and tracked accurately. While the wearable device teaches sensors and automatic detection and collection of data as input, there are also alternative options interaction and manual input.

The wearable device taught by the present invention uses voice recognition/control and touch, with machine learning and predictive analytics, to monitor the occurrence and duration of postnatal and postpartum care and development events. The wearable device will track, remind/alert, correlate, predict, suggest, document, integrate and transmit past, present and future events to alleviate the burden that users experience with management by manual or less comprehensive methods.

Now focusing on the physical features of the wearable device, the wearable device includes a touch screen made from passive or active-matrix organic light-emitting diodes.

The wearable device is controlled by voice recognition, which is initially set up when a user types and then speaks several words/phrases that will be frequently used for each action or using preset words. Most speech/voice recognition applications select the few words that the user repeats to calibrate a device, and the present invention will function in a similar manner. The calibration is similar, however where most devices provide the words to repeat, the present invention allows the user to select from a list and/or designate alternate words.

The device will use a phonetic approach and will calibrate voice recognition by action to improve accuracy. The user can designate a specific set of words/phrases for each action. For example, breast feeding is calibrated with a different set of words than diaper changing, and the set of words can vary from user to user.

The device can be controlled by voice, which is ideal when caring for a baby where the caregiver is often placed in situations with limited hand mobility such as diaper changing, feeding, or even playing.

Machine Learning/Predictive Analytics provides activity recognition and serves to correlate baby and mom/dad data to better predict events using statistical analysis/trending techniques.

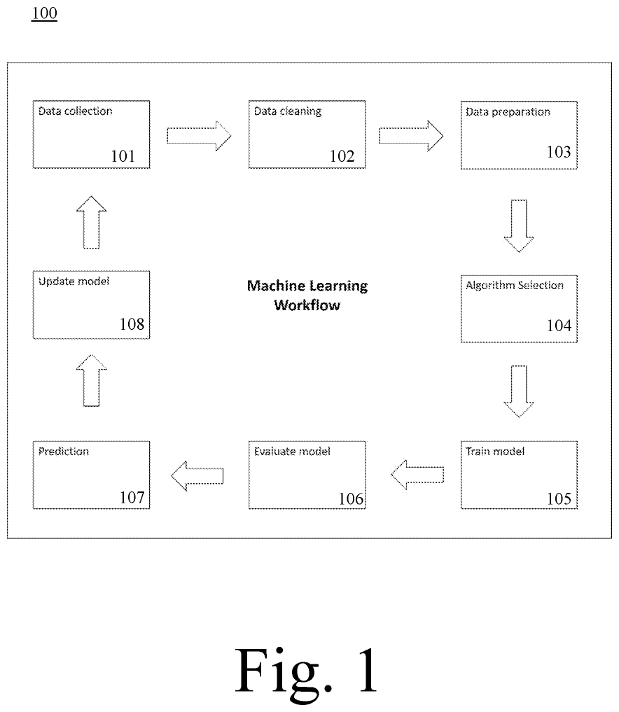

As shown in FIG. 1, a typical machine learning workflow 100 taught and used by the present invention is illustrated. Here, a circular process is used whereby data collected 101 is run through a data cleaning process 102 and sent to a data preparation module 103. Prepared data is then sent through an algorithm selection module 104 and then to a training module 105. An evaluation module 106 then takes the data from the training module 105 and generates a prediction 107 as the output, which is then used to update the model 108. The process then repeats for the next set up data collected.

Combinations of actions such as when a mother nurses a left breast while pumping a right breast can also be tracked. Tracking can also be done by independent timers for each activity, and not simply as the double entry of activities over a single tracked period of time.

As with other devices, the present invention will use a series of vibration and color light/LED notifications, which may be on screen or external to the device display. Touch control is ideal when caring for a baby during noise-sensitive events, such as when a baby is sleeping and will be provided. The device of the present invention will also include and incorporate the following known features: a backlit screen, a camera, flashlight with a nightlight setting, a keyboard, a basic account, a clock, a timer and alarm, a counter/movement tracker, a calendar, data storage on a memory card, local memory, or wirelessly to cloud storage, email, individual or group messaging/Community chat, and WIFI.

The wearable device of the present invention will also utilize and provide; baby cam feed capability, a point of care adapter-ready to accept devices for providing lateral flow, colorimetric, electrochemical, fluorometric, otoscopic, microscopic, a timer/alarm that can set a schedule, be adjusted if an event is missed or shifted, or include an entire series or specific event; a counter/tracker providing automatic record of entries; a calendar for providing specific reminders such as baby milestone photos, baby appointments, and mom appointments.

With respect to files and document tracking, the wearable device of the present invention allows for entering and storing documents/notes such as one or more health insurance cards, list, such as baby care shopping lists, a filing folder system for organization, the integration of multiple user data, transmission and export of data to maintain and share records with health providers or other third parties.

Baby activity tracking includes but is not limited to the following: diaper change with wet/poop indications, nursing, bottle feeding, pumping, sleep, nap, bath, burp, vitamins, meds, meals, and tummy time.

Baby health tracking includes but is not limited to the following: crying, vomiting, diarrhea, fever, cough, runny nose, and sneezing.

Baby milestone tracking includes but is not limited to the following: follow objects, grabbing objects, hold head during tummy time, standing on lap, brings hand to mouth, cooing, pushes feet against resistance, smiles, laughs, blowing raspberries, brings hand to mouth, babbling sounds, plays with toys, picks up objects, sits up supported by pillows, rolling over (tummy to back), holding up head, eating solids, transfer objects between hands, exploring objects with mouth, rolling over (tummy to back and back to tummy), tracks objects with eyes, understands name, understands no, saying sounds like mama, reacts to emotion in voice, loves playing with you, sitting unassisted, crawling, attached to toy, pulls up self to stand, grasp smaller objects, waves, use fingers to point, afraid of strangers/clingy with parents, can get into sitting position without support, understands baby sign language, communicates with gestures, looks at objects when named, stands for a few seconds, cruises with toys, says first words, follows simple directions, walking without support, exploring objects by banging, imitates words, remembers where objects are hidden, uses sippy cup, uses gestures, preference for certain people, separation anxiety, and holds marker and scribbles.

User activity tracking includes but is not limited to the following: bath, exercise, sleep, fluids/water, me time, laundry, and cleaning.

User health tracking includes but is not limited to the following: meal/calories, meds, vitals monitor including but not limited to blood pressure, heart rate, oxygen, temperature, and sleep, and pain.

With respect to functionality, the wearable device will track, remind, predict, suggest, integrate, correlate, document and transmit events during postnatal and postpartum care.

In a preferred embodiment, the wearable device is a smartwatch with voice control/touch screen input where a user can control counters, timers, etc. using voice commands and/or touch input.

The wearable device of the present invention teaches simple/minimal steps for setup and operation where multiple postnatal actions such as: nursing, feeding, sleeping, diaper change, bath, medicine administration, etc. can be tracked. Multiple postpartum actions such as eating, fluid intake, exercise, sleep, etc. can also be tracked.

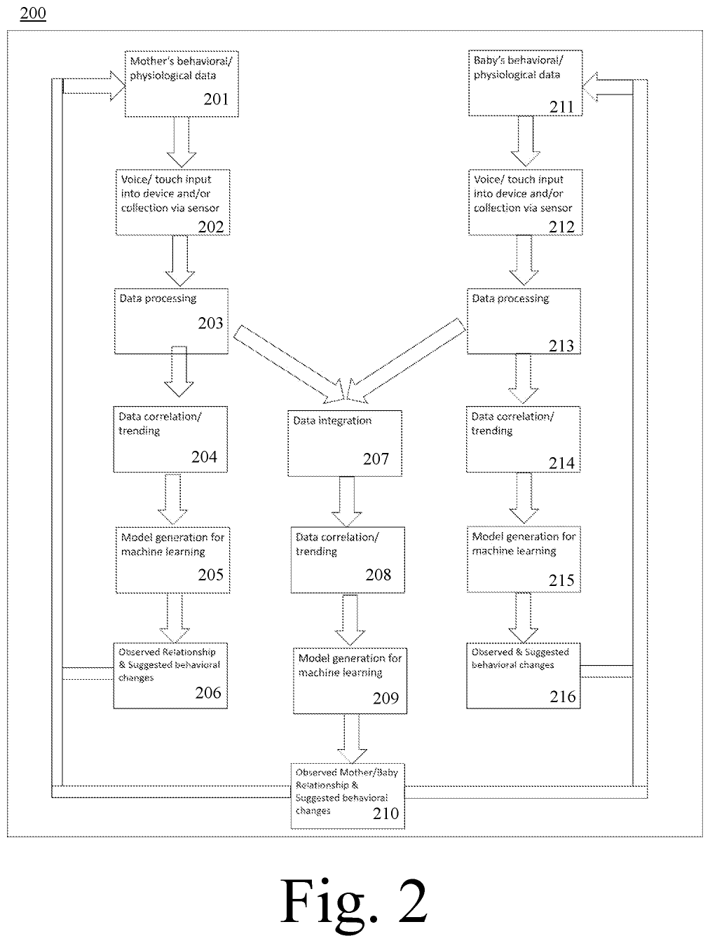

Now referring to FIG. 2 a general workflow 200 taught by the present invention is illustrated. Here mother and baby behavioral/physiological data 201 and 211 is inputted to the wearable device(s) via voice or touch input and/or collection via a sensor 202 and 212. The individual data 201 and 211 is then processed and integrated together 207 as well as individually 203 and 213 being sent to a data correlation/trending module 204, 208, and 214 for analysis. The baby and mother can each have their own data correlation/trending module for analysis using machine learning (ML) 205 and 215 which generates specific, individual observed relationship and suggested behavioral change information 206 and 216. The integrated data 207 also undergoes analysis through a separate data correlation and trending module 208, using machine learning (ML) 209. The correlated/trending integrated data as well as the observed relationship and suggested behavioral changes 206 and 216 for both the mother and baby are finally combined into an observed mother/baby relationship and suggested behavioral changes output 210.

It should be noted that the present invention can be applied to one or more babies/newborns, and one or more caregivers, where “mother” is simply the name given to represent the caregiver in a single caregiver and single baby embodiment of the invention. Again, the invention can include multiple newborns/babies, mother/father caregivers, wet nurses, nannies, grandparents, or another combination of people providing care to one or more newborns or babies.

The postnatal and postpartum data will be integrated together and correlated. For instance, if breast milk volume increases when mother fluid intake increases.

The wearable device of the present invention will correlate baby and mom/dad data to generate trends and make suggestions for actions. Examples include: The baby drank less milk that day than previous days, it will suggest to add an additional feeding; If the baby's crying is correlated with not burping the baby, the watch will remind the mom/dad to burp baby after feeding; When a mom/dad sleeps less than 8 hours at night due to activities like pumping, the watch will suggest that the mom/dad naps during the next baby nap; and When the baby has fewer wet diapers, the watch will suggest that the mom increases fluids to increase breast milk volume.

The wearable device of the present invention supports multiple users with integrated data collection where a baby's data from mom's watch/account will be integrated with baby's data from dad's watch/account. A user can view the history of an event by pushing an icon on the watch screen or using a voice command. The user can input, through voice or touch, data throughout the day.

The backend of the wearable device will compute and record the event details with the date/time it occurred and duration. A duration timer can be set to alert the user of the next event.

There's an option to input details during each event occurrence.

There's a calendar for reminders.

There's a notepad for notes about milestones and other information.

An algorithm will generate suggestive care based on event history.

Data can be exported in table or graph form to an email, text, and cloud drive.

Messaging between users (individually, group, and community chat) is also supported.

In one exemplary operation, to set up the voice recognition of the breastfeeding timer, the watch will ask the user to type in and speak several words they will use to start the timer. For instance: Start, begin, timer, breastfeed and nurse. These words will be used to set up the voice control. The user can start and stop a timer. The duration is recorded, and the event is tallied/totaled in a counter. For example: To record every time a mother feeds a baby and how long the feeding lasted.

A timer can be set for breast feeding on the left side, while also pumping on the left side. The user will state “Watch, start breast feeding left timer. Start pumping right timer.” The timer will start for each. When the user finishes, the user will state “Watch, stop breast feeding left timer. Stop pumping right timer.” The timers will stop. They can be stopped sequentially or after different durations.

A timer can be set for a specified period of time from the last event. For example: 45 minutes from entered Action #1. The user can end/accept the end timer or extend it. The timer will make an audible sound or can be muted to display a visual notification.

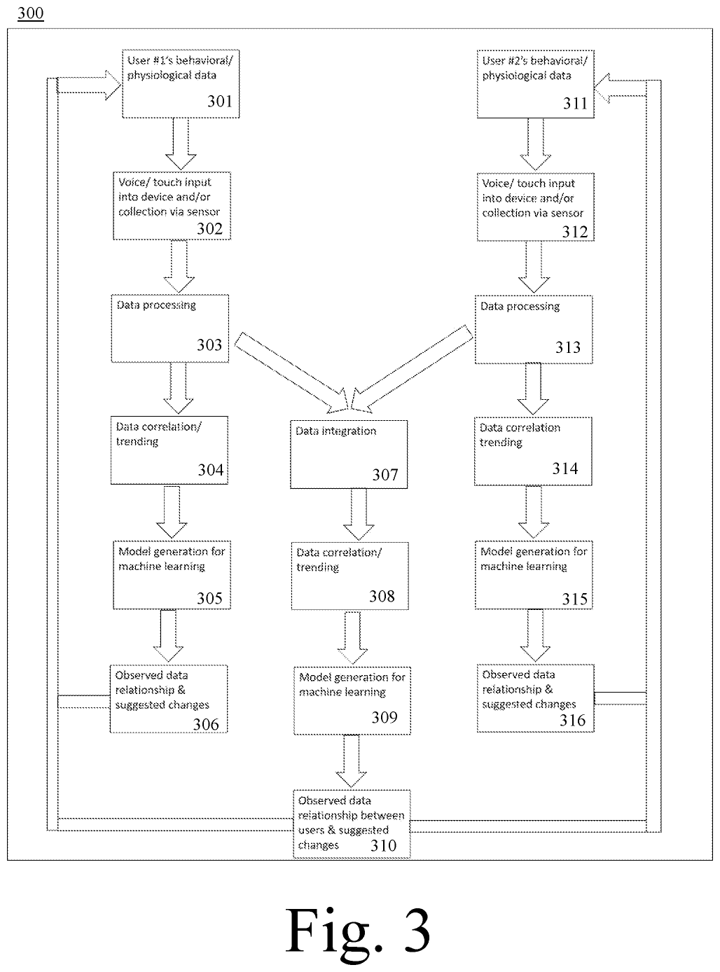

FIG. 3 illustrates a generic workflow 300 taught by the present invention. In this embodiment a first and second user behavioral/physiological data 301 and 311 is inputted to the wearable device(s) via voice or touch input and/or collection via a sensor 302 and 312. The individual data is then processed 303 and 313 and integrated together 307 as well as individually being sent to a data correlation/trending module 304 and 314 for analysis. The baby and mother can each have their own data correlation/trending module 304 and 314 for analysis using machine learning (ML) 305 and 315 which generates specific, individual observed relationship and suggested behavioral change information 306 and 316. The integrated data 307 also undergoes analysis through a separate data correlation and trending module 308, using machine learning (ML) 309. The correlated/trending integrated data 309 as well as the observed relationship and suggested behavioral changes for both the mother and baby 306 and 316 are finally combined into an observed relationship and suggested behavioral changes output 310 between the users.

An alarm can be set for a specified time. For example: 4 μm. The user can accept or cancel the timer. The alarm will make an audible sound or can be muted to display a visual notification.

The user can monitor her health throughout the day by setting an alarm to remind them. The user can also record their vitals at a random time.

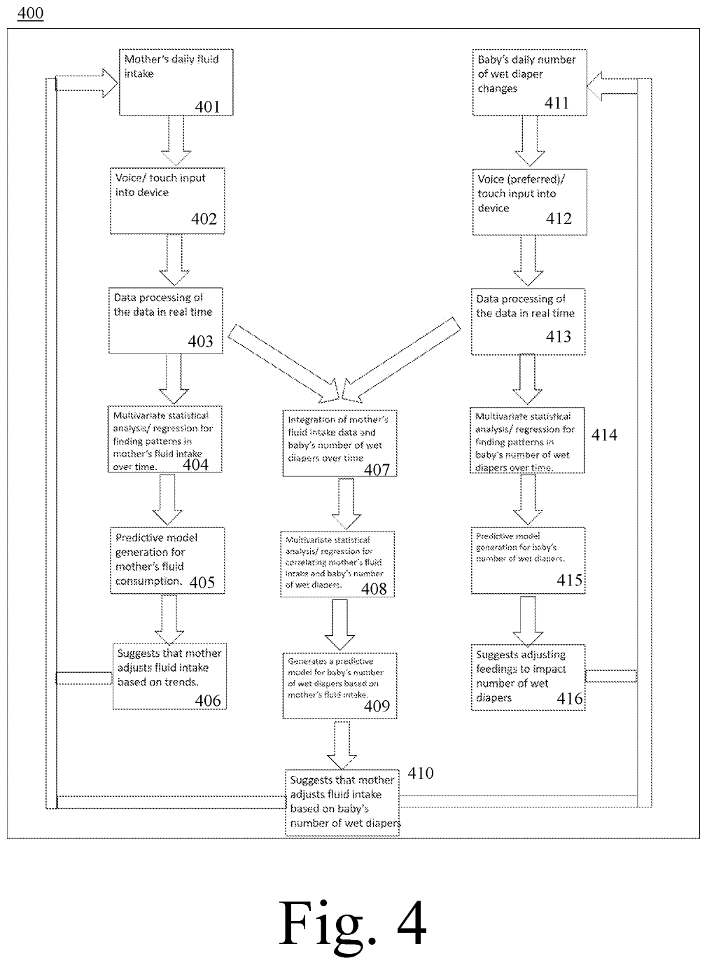

Finally, FIG. 4 illustrates a specific workflow 400 taught by the present invention with respect to a mother's daily fluid intake 401 and the relationship between the baby's fluid output 411. In this embodiment a mother's daily fluid intake 401 and a baby's daily number of wet diaper changes 411 is inputted to the wearable device(s) via voice or touch input and/or collection via a sensor 402 and 412. The individual data is then processed in real time 403 and 413 and integrated together 407 as well as individually being sent to a data module 404, 408, and 414 for individual multivariate statistical analysis/regression for finding pattern in a mother's fluid intake over time.

The baby and mother can each have their own, individual data subjected to a multivariate statistical analysis/regression, using machine learning (ML), for finding pattern in a mother's fluid intake over time and for finding patterns in a baby's number of wet diapers over time. A predictive model 405 and 415 then generates a prediction output for the mother's fluid consumption as well as for a baby's number of wet diapers and makes suggestions 406 and 416 whether a mother should adjust their fluid intake based on the trends and predictions as well as whether to adjust feedings to impact the number of wet diapers being experienced and predicted to be experienced by the baby.

The integrated data 407 is subjected to a multivariate statistical analysis/regression, using machine learning (ML) 408, for correlating a mother's fluid intake and a baby's number of wet diapers. A predictive model 409 generates a prediction for the baby's number of wet diapers based on the mother's fluid intake. Finally, the individual suggestion 406 and 416 from the individual data is combined with the suggestion generated from the integrated data 409 to generate output suggestions 410 to provide any suggested mother fluid intake adjustments that should be made based on the baby's number of wet diapers.

Although FIG. 4 illustrates and provides one example of the application for the method and process for analyzing the input date as it relates to a baby and caregiver, the present invention should not be limited to this one exemplary application relating fluid intake to wet diapers, and it should be appreciated that that inventors can and will apply the same method and process disclosed to other related data and cases.

Additionally, although this exemplary illustration is between a mother and one baby, it should also be appreciated that it is not limited to a mother or a single caregiver, and also not limited to a single baby/newborn and that any combination of plurality of caregivers and baby/newborns is applicable as well as the number of wearable devices for providing sensors and the auto collection and entry of data can be utilized.

In one alternative embodiment, athletes who play a team sport such as relay track can track their physiological (i.e. heart rate, blood pressure, blood oxygen, sleep), behavioral (i.e. calorie intake, fluid intake), and physical (i.e. steps, speed) events to correlate the user's data with another user's or the team's data. For example, this would result in suggested changes in behavior physiological and behavioral of a user(s) to determine the optimal physical data for a team.

In another embodiment, workers (i.e. health care and lab) can monitor their behavioral (i.e. shift time, fluid intake, meals), environmental (i.e. exposure risks), and physiological (i.e. sleep, symptoms, temperature, heart rate, blood pressure, blood oxygen) events to correlate the user's data with another user's or the team's data. For example, this would result in suggested changes to behavior of a user(s) based on the physiological data of another user or the team.

In still another embodiment, emergency workers (as well as lab and industrial workers) can monitor their environmental/chemical/biological (i.e. activities, exposure) and physiological (i.e. symptoms, temperature, heart rate, blood pressure, blood oxygen) events to correlate the user's physiological data with another user's or the team's physiological and environmental/chemical/biological data. For example, this would result in suggested exposure risk (negative physiological events) of users based on environmental/chemical/biological data of other users.

Thus, it is appreciated that the optimum dimensional relationships for the parts of the invention, to include variation in size, materials, shape, form, function, and manner of operation, assembly, and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. A wearable device for tracking, reminding/alerting, correlating, predicting, suggesting, documenting, integrating, and transmitting postnatal and postpartum care and development events, comprising
a touch screen providing touch control and input,
a microphone providing voice recognition/control and input;
machine learning and predictive analytics, to monitor the occurrence and duration of postnatal and postpartum care and development events; and
an application software to track, remind/alert, correlate, predict, suggest, document, integrate and transmit past, present and future events to alleviate the burden that users experience with management by manual or less comprehensive methods.

2. The wearable device of claim 1, wherein the wearable device is a smartwatch.

3. The wearable device of claim 1, wherein the wearable device is a fitness tracker.

4. The wearable device of claim 1, wherein the touch screen is made from passive or active-matrix organic light-emitting diodes.

5. The wearable device of claim 1, wherein
the wearable device is controlled by voice recognition, which initially sets up wake word and/or action words by speaking several words/phrases that will be used.

6. The wearable device of claim 5, wherein
the device uses a phonetic or universal allowing preset commands approach and will calibrate voice recognition by action to improve accuracy;
the user to selects from
a list and/or designate alternate words,
a pre-set words or
a specific set of words/phrases for each action can be designated; and
the set of words can vary from user to user.

7. The wearable device of claim 5, wherein
machine learning/predictive analytics provides activity recognition and serves to correlate baby and mom/dad/data to better predict events using statistical analysis/trending techniques.

8. The wearable device of claim 1, wherein
a series of vibration and color light/LED notifications, which may be on screen or external to the device display.

9. The wearable device of claim 1, further comprising
a backlit screen,
a camera,
flashlight with a nightlight setting,
a keyboard,
a basic account,
a clock,
a timer and alarm,
a counter/movement tracker,
a calendar,
data storage on a memory card, local memory, or wirelessly to cloud storage,
email,
individual or group messaging/Community chat, and
WIFI.

10. The wearable device of claim 1, further comprising
a baby cam feed capability,
a point of care adapter-ready to accept devices for providing
lateral flow, colorimetric, electrochemical, fluorometric, otoscopic, microscopic,
a timer/alarm that can set a schedule, be adjusted if an event is missed or shifted, or include an entire series or specific event;
a counter/tracker providing automatic record of entries;
a calendar for providing specific reminders such as baby milestone photos, baby appointments, and mom appointments.

11. The wearable device of claim 9, further comprising
with respect to files and document tracking,
entering and storing documents/notes such as one or more health insurance cards,
lists, such as baby care shopping lists,
a filing folder system for organization, the integration of multiple user data, transmission and export of data to maintain and share records with health providers or other third parties.

12. The wearable device of claim 1, further comprising
Baby activity tracking;
Baby health tracking;
Baby milestone tracking;
User activity tracking; and
User health tracking.

13. The wearable device of claim 12, wherein
with respect to functionality, the wearable device will
track, remind, predict, integrate, correlate, predict, suggest, document and transmit events during postnatal and postpartum care.

14. The wearable device of claim 12, wherein
multiple postnatal actions such as: nursing, feeding, sleeping, diaper change, bath, milestones, symptoms, health events, medicine administration, etc. are tracked; and
multiple postpartum actions such as eating, fluid intake, pumping, exercise, sleep, symptoms, and health events are tracked.

15. A method for tracking, reminding/alerting, correlating, predicting, suggesting, documenting, integrating, and transmitting postnatal and postpartum care and development events in a wearable device, comprising
a circular process whereby data collected is run through a data cleaning process and sent to a data preparation module;
prepared data is then sent through an algorithm selection module and then to a training module;
an evaluation module then takes the data from the training module and generates a prediction as the output, which is then used to update the model; and
the process then repeats for the next set up data collected.

16. The method of claim 15, further comprising wherein
combinations of actions are also be tracked; and
tracking is done by independent timers for each activity.

17. The method of claim 16, further comprising wherein
athletes who play a team sport can track their
physiological heart rate, blood pressure, blood oxygen, sleep),
behavioral calorie intake, fluid intake, and
physical steps, speed,
events to correlate the user's data with another user's or the team's data.

18. The method of claim 16, further comprising wherein
workers monitor
behavioral shift time, fluid intake, meals,
environmental exposure risks, and
physiological sleep, symptoms, temperature, heart rate, blood pressure, blood oxygen
events to correlate the user's data with another user's or the team's data.

19. The method of claim 16, further comprising wherein
emergency, laboratory, and industrial workers monitor
environmental/chemical/biological activities, exposure, and
physiological symptoms, temperature, heart rate, blood pressure, blood oxygen
events to correlate the user's physiological data with another user's or the team's physiological and environmental/chemical/biological data.

20. The method of claim 15, wherein
mother and baby behavioral/physiological data is inputted to the wearable device(s) via voice or touch input and/or collection via a sensor;
the individual data is then processed and integrated together as well as individually being sent to a data correlation/trending module for analysis;
the baby and mother can each have their own data correlation/trending module for analysis using machine learning (ML) which generates specific, individual observed relationship and suggested behavioral change information;
the integrated data also undergoes analysis through a separate data correlation and trending module, using machine learning (ML); and
the correlated/trending integrated data as well as the observed relationship and suggested behavioral changes for both the mother and baby are finally combined into an observed mother/baby relationship and suggested behavioral changes output.

21. The method of claim 15, wherein
the wearable device of the present invention supports multiple users with integrated data collection wherein
a baby's data from mom's watch/account will be integrated with baby's data from dad's watch/account;
a user can view the history of an event by pushing an icon on the watch screen or using a voice command; and
the user can input, through voice or touch, data throughout the day.

22. The method of claim 15, wherein
the backend of the wearable device will compute and record the event details with the date/time it occurred and duration;
a duration timer can be set to alert the user of the next event;
there's an option to input details during each event occurrence;
there's a calendar for reminders;
there's a notepad for notes about milestones and other information;
an algorithm will generate suggestive care based on event history;
data can be exported in table or graph form to an email, text, and cloud drive; and
messaging between users (individually, group, and community chat) is supported.

23. The method of claim 15, wherein
first and second user behavioral/physiological data is inputted to the wearable device(s) via voice or touch input and/or collection via a sensor;
the individual data is then processed and integrated together as well as individually being sent to a data correlation/trending module for analysis;
the baby and mother can each have their own data correlation/trending module for analysis using machine learning (ML) which generates specific, individual observed relationship and suggested behavioral change information;
the integrated data also undergoes analysis through a separate data correlation and trending module, using machine learning (ML); and
the correlated/trending integrated data as well as the observed relationship and suggested behavioral changes for both the mother and baby are finally combined into an observed relationship and suggested behavioral changes output between the users.

24. The method of claim 15, wherein
an alarm can be set for a specified time;
the user can monitor her health throughout the day by setting an alarm to remind them; and
the user can also record their vitals at a random time.
